(19) United States
(12) Patent Application Publication
(10) Pub. No.: US 2013/0032147 A1
(43) Pub. Date: Feb. 7, 2013

(54) METHOD AND SYSTEM FOR VISUALIZING MECHANICAL VENTILATION INFORMATION

(71) Applicants: Scott William Robinson, Bayside, WI (US); Andreas Tzanetakis, Helsinki (FI); Nathaniel Brazy, Madison, WI (US); Bryan Powell, Longmont, CO (US)

(72) Inventors: Scott William Robinson, Bayside, WI (US); Andreas Tzanetakis, Helsinki (FI); Nathaniel Brazy, Madison, WI (US); Bryan Powell, Longmont, CO (US)

(73) Assignee: General Electric Company, Schenectady, NY (US)

(21) Appl. No.: 13/198,514

(22) Filed: Aug. 4, 2011

(51) Int. Cl. A61M 16/00

(52) U.S. Cl. 128/204.18

(57) ABSTRACT

A medical system having a ventilator is provided. The medical system includes a memory storing data related to patient and ventilator parameters. A processor is programmed to organize, in time, the data related to the patient and ventilator parameters. A viewer displays the patient and ventilator parameters for an overall monitoring time period on a timeline. A reference marker is displayed on the timeline to select a segment of the patient and ventilator parameters corresponding to a past monitoring time, wherein the segment selected represents a time segment within the overall monitoring time period. The patient and ventilator parameters corresponding to the time segment are displayed.

### BACKGROUND OF THE INVENTION

The subject matter disclosed herein relates generally to respiratory care systems, and more particularly, to mandatory mechanical ventilation systems.

When patients are medically unable to breathe on their own, mechanical or forced ventilators can sustain life by providing requisite pulmonary gas exchanges for the patients. For example, conventional ventilators typically include electronic and pneumatic systems that control the pressure, flow rates, and/or volume of gases delivered to, and extracted from, patients needing medical respiratory assistance. Such control systems often include numerous user controls, such as knobs, dials, switches, and the like, for interfacing with treating clinicians, who support the patient's breathing by adjusting the pressure, flow rates, and/or volume of the patient's pulmonary gas exchanges, particularly as the condition and/or status of the patient changes. These parameter adjustments are challenging to control accurately, particularly using these conventional systems.

With respect to ventilation, this is a complex process of delivering oxygen to, and removing carbon dioxide from, alveoli within patients' lungs. Thus, conventional ventilators, particularly controlled mechanical ventilation (CMV) systems, include inputs that allow operating clinicians to select and use several modes of ventilation, either individually and/or in various combinations, using different ventilator setting controls. These mechanical ventilators have become increasingly sophisticated and complex, due in part to enhanced understandings of lung pathophysiology. Accordingly, many conventional ventilators are microprocessor-based and equipped with sensors that monitor patient pressure, flow rates, and/or volumes of gases, and then drive automated responses in response thereto. However, as these ventilators become more complicated and provide more options, the number and risk of potentially dangerous clinical decisions increases as well. Thus, clinicians often operate expensive, sophisticated machines, yet few follow clear, concise, and/or consistent guidelines for maximal use thereof. For example, numeric information may be collected into tables and spreadsheets. Accordingly, clinicians may be required to search through pages of tabular data searching for critical numeric indicators of patient status. As a result, setting, monitoring, and interpreting ventilator parameters may be reduced to empirical judgment, resulting in less than optimal treatment. For example, tracking past patient data along with present patient data and future extrapolated data may be time consuming.

Thus, the overall effectiveness of assisted ventilation ultimately depends on mechanical, technical, and physiological factors, with the clinician-ventilator-patient interface playing an important role. For example, clinicians often need to observe and control several factors to optimize the volume of air that is appropriate given the particular patient. However, it is often difficult for clinicians to observe and control these several factors at the same time.

### SUMMARY OF THE INVENTION

In one embodiment, a medical system having a ventilator is provided. The medical system includes a memory storing data related to patient and ventilator parameters. A processor is programmed to organize, in time, the data related to the patient and ventilator parameters. A viewer displays the patient and ventilator parameters for an overall monitoring time period on a timeline. A reference marker is displayed on the timeline to select a segment of the patient and ventilator parameters corresponding to a past monitoring time, wherein the segment selected represents a time segment within the overall monitoring time period. The patient and ventilator parameters corresponding to the time segment are displayed.

In another embodiment, a method for presenting ventilator data is provided. The method includes storing data related to patient and ventilator parameters. The data related to the patient and ventilator parameters is organized, in time. The patient ad ventilator parameters are displayed for an overall monitoring time period on a timeline. A reference marker is displayed on the timeline to select a segment of the patient and ventilator parameters corresponding to a past monitoring time. The segment selected represents a time segment within the overall monitoring time period. The patient and ventilator parameters corresponding to the time segment are displayed.

In yet another embodiment, a non-transitory computer readable storage medium for displaying ventilator information using a processor is provided. The non-transitory computer readable storage medium includes instructions to command the processor to store data related to patient and ventilator parameters, and organize, in time, the data related to the patient and ventilator parameters. The processor is also commanded to display the patient and ventilator parameters for an overall monitoring time period on a timeline, and display a reference marker on the timeline to select a segment of the patient and ventilator parameters corresponding to a past monitoring time. The segment selected represents a time segment within the overall monitoring time period. The processor is also commanded to display the patient and ventilator parameters corresponding to the time segment.

### BRIEF DESCRIPTION OF THE DRAWINGS

FIG. 1 is a simplified block diagram of a medical system in accordance with various embodiments.

FIG. 2 illustrates a patient ventilator parameter display formed in accordance with an embodiment.

FIG. 3 is an exemplary view of a screen including the patient ventilator parameter display.

FIG. 4 is another exemplary view of the screen shown in FIG. 3.

FIG. 5 is another exemplary view of a screen shown in FIG. 3.

FIG. 6 is another exemplary view of a screen shown in FIG. 3.

FIG. 7 is another exemplary view of a screen shown in FIG. 3.

FIG. 8 is a flowchart illustrating a method for displaying data from a ventilator.

### DETAILED DESCRIPTION OF THE INVENTION

The foregoing summary, as well as the following detailed description of certain embodiments, will be better understood when read in conjunction with the appended drawings. The figures illustrate diagrams of the functional blocks of various embodiments. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like),or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

The various embodiments provide a user interface for a patient ventilator. In particular, various embodiments provide a time based viewer providing a user interface for a patient ventilator. Specifically, the viewer displays a visual representation of patient ventilation parameters. For example, the patient ventilation parameters may be related to a patient's breathing ability while on a ventilator. At least one technical effect of various embodiments is the ability to monitor the patient ventilation parameters in the past and present, and provide clinical decision support for future patient treatment.

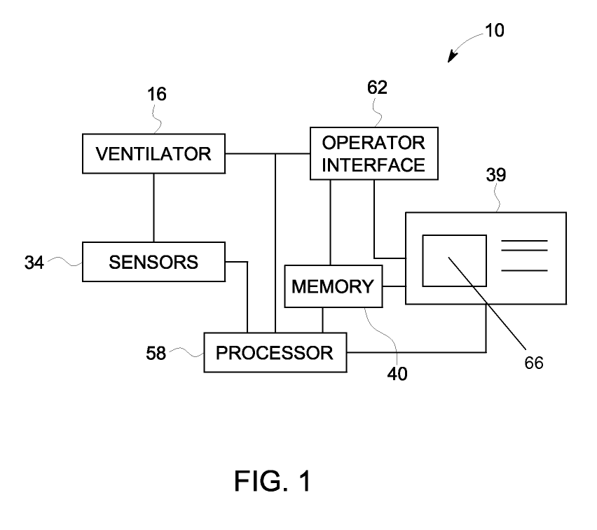

The viewer may be implemented in a patient monitoring system providing mechanical ventilation. For example, the viewer may be implemented in a medical system 10 as illustrated in FIG. 1. The medical system 10 in various embodiments provides for mechanically ventilating a patient (not shown). The medical system 10 also provides for visualization of the patient ventilation (or ventilator) parameters for use in controlling a ventilator 16 based on displayed compliance data in combination with measurement data from one or more sensors 34. The ventilator may be controlled via an operator interface 62 by a clinician viewing the visualized patient and ventilator parameter data, which may be multiple data types displayed concurrently on a monitor 38 to allow a user to view a balance between at least some of the patient ventilation parameters. For example, a user may view a plurality of different types of data acquired at different times. A processor 58, for example, a processing subsystem of the medical system 10 may process received measurements from the sensors 34 and other compliance information as described herein to update the Monitor 38 with the patient ventilation parameter data. The medical system 10 may include a memory 40 for storing data related to the patient ventilator parameters. The processor 58 may be programmed to organize data related to the patient ventilator parameters.

The ventilator 16 further can receive inputs from the sensors 34 associated with the patient (e.g., coupled to the patient) and/or the ventilator 16 at the processor 58 for subsequent processing thereof, and which can be displayed on the monitor 38. Representative data received from the sensors 34 can include, for example, inspiratory time (T_{I}), expiratory time (T_{E}), natural exhalation time (T_{EXH}), respiratory rates (f), I:E ratios, positive end expiratory pressure (PEEP), fractional inspired oxygen (F_{I}O₂), fractional expired oxygen (F_{E}0₂), breathing gas flow (F), tidal volumes (V_{T}), temperatures (T), airway pressures (P_{aw}), arterial blood oxygen saturation levels (SₐO₂), blood pressure information (BP), pulse rates (PR), pulse oximetry levels (SₚO₂), exhaled CO₂ levels (F_{ET}CO₂), concentration of inspired inhalation anesthetic agent (C_{I} agent), concentration of expired inhalation anesthetic agent (C_{E} agent), arterial blood oxygen partial pressure (PₐO₂), arterial carbon dioxide partial pressure (PₐCO₂), and the like.

The components are functionally depicted for illustration, wherein various components thereof can also be integrated and/or separated, as needed and/or desired. Other functional components, for example, one or more power supplies for the medical system 10 and/or ventilator 16 may be provided.

Various embodiments provide a viewer 66 that is a user interface tool for the medical system 10, and specifically for controlling operation of the ventilator 16. For example, the viewer 66 enables a user, such as a clinician, to balance and evaluate the patient ventilation parameters in the past, present, and future. Thus, the viewer 66 allows control of one more ventilator parameters or settings based on displayed information, which may be related in part to patient physiology. Accordingly, the various embodiments allow clinicians to control patient ventilation parameters throughout the respiratory cycle of the patient and enables ventilation control or treatments to be individually controlled (e.g., optimized) for patients subject to controlled mechanical ventilation (CMV).

The ventilator 16 may be used to measure different conditions or parameters, for example, inspiratory time (T_{I}). T_{I} is the amount of time, measured in seconds, set on the ventilator 16 by the clinician, lasting from the beginning of the inspiration of the patient to the beginning of the expiration of the patient. The ventilator 16 may also measure other parameters, for example, expiratory time (T_{E}), I:E ratios (e.g. the ratios between T_{I} and T_{E}), natural exhalation time (T_{EXH}), positive end expiratory pressure (PEEP), tidal volume (V_{T}), respiratory rate (f), and/or any other parameters generally measured by a ventilator.

The viewer 66 may be used to adjust the settings of the ventilator 16 to balance patient ventilation parameters. In particular, using the visualizations provided by various embodiments, the degree to which an ideal balance between the patient ventilation parameters has been achieved may be visually observed and determined, thereby allowing a user, such as a clinician to adjust the settings of the ventilator 16, such as to change the initial settings of the patient ventilation parameters.

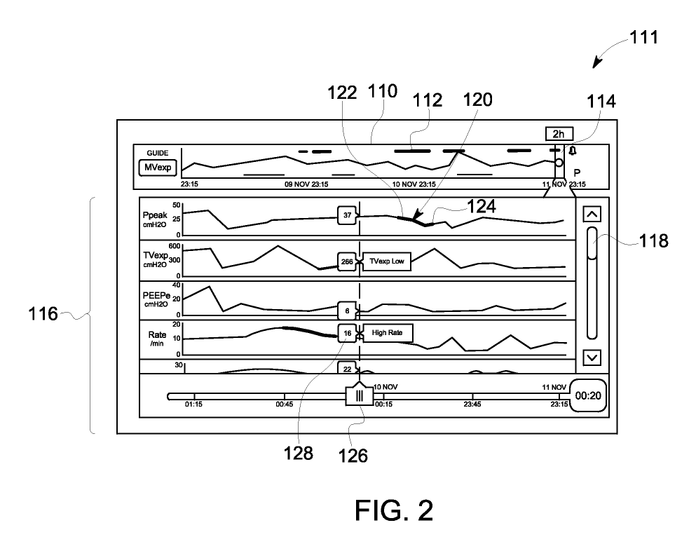

FIG. 2 illustrates a patient ventilator parameter display 111 including a master timeline 110 that provides a constant universal time reference of a general patient status. In one embodiment, the master timeline 110 is always displayed. In one embodiment, all views tied to a particular time period (e.g. alarm data) are always displayed on the master timeline 110. The master timeline 110 may represent an overall monitoring period for the patient. The patient ventilator parameter display 111 may be displayed on the viewer 66. In the illustrated embodiment, the master timeline 110 displays expiration minute volume (MVexp) of the patient. Alternatively, the master timeline 110 may display any patient ventilator parameter. In an exemplary embodiment, the master timeline 110 represents a past patient ventilator parameter indicative of data acquired by the ventilator 16 (shown in FIG. 1). The master timeline 110 displays the patient ventilator parameter over an extended period of time (e.g. the time that the patient has been on the ventilator 16).

The master timeline 110 may include alert notifications 112 in the form of horizontal lines displayed on the master timeline 110 at the time that an alert occurred. The alert notifications represent a patient ventilator parameter falling outside of a compliance range for the parameter, wherein the compliance range represents a desired output for the parameter. Alternatively, the alert notifications 112 may take the form of a symbol or the like. In one embodiment, the alert notifications 112 may be color coded to indicate a particular patient ventilator parameter that triggered an alert. The master timeline 110 includes a master reference marker 114 that enables an operator to focus on a particular time segment of the master timeline 110. In particular, the master reference marker 114 may be used to select a time segment within the extended period of time displayed on the master timeline 110. For example, the master reference marker 114 may be configured to select a one hour time segment, a one day time segment, a one week time segment, or the like. A length of the time segment may be adjusted by an operator. The master reference marker 114 may be moved along the master timeline 110 to select a portion of the patient ventilator parameters displayed on the master timeline 110. For example, the master reference marker 114 may be moved using a touch screen display, a dial, a keyboard, or the like.

Thus, in various embodiments, as described in more detail herein and with reference to the Figures described below, an operator is able to switch between different views, for example, of trend data, while maintaining the same time reference as shown in the master timeline 110. It should be noted that like numerals represent like parts in the Figures.

Local timelines 116 display the portion of the master timeline 110 selected with the master reference marker 114. The local timelines 116 display patient ventilator parameters during the time segment selected on the master timeline 110. For example, in the illustrated embodiment, the local timelines 116 display a two hour time segment of the master timeline 110. In an exemplary embodiment, the local timelines 116 display past patient ventilator parameters indicative of data acquired by the ventilator 16. Any number of local timelines 116 may be displayed to illustrate any number of patient ventilator parameters. A scroll bar 118 may provided to scroll through the local timelines 116. Alert notifications 120 may also be represented on the local timelines 116. For example, if a patient ventilator parameter falls outside of a compliance range, an alert notification 120 may be displayed on the local timeline 116 representing the parameter. The alert notification 120 may include a colored portion of the local timeline 116 from a starting point 122, when the parameter falls outside of the compliance range, to an ending point 124, when the parameter returns within the compliance range. Alternatively, the alert notification 120 may be displayed as a line above or below the corresponding local timeline 116 and extending from the starting point 122 to the ending point 124. In other embodiments, the alert notification 120 may be displayed as a symbol or the like.

A local reference marker 126 is displayed below the local timelines 116. The local reference marker 126 is moveable along the local timelines 116 to display data from the patient ventilator parameters based on data points selected on the local timelines 116 with the local reference marker 126. The local reference marker 126 may be Moved using a touch screen display, a knob, a keyboard, a cursor, or the like. Numerical data 128 related to each of the patient ventilator parameters is displayed based on a position of the local reference marker 126. As the local reference marker 126 is moved, the numerical data 128 updates accordingly. In one embodiment, a cursor may be provided to select a portion of any local timeline 116. Upon selecting a portion of a local timeline 116, the corresponding numerical data 128 is displayed.

The patient ventilator parameter display 111 provides visualization of expansive data (e.g. data over extended periods of time) that may otherwise be cumbersome to review. The display 111 implements historic trending that translates the patient ventilator parameter data into simplified graphic symbols that are organized into logical groups and made available (on the display 111) when selected by an operator. Historic trending translates selected values of the patient ventilator parameters into linear trend lines on the timelines 116. Additionally, alarms and/or events are translated into graphic symbols that complement the trends on the timeline 116.

In an exemplary embodiment, to analyze any moment on the timelines 116, the operator moves the local reference marker 126 over the timelines 116, alarms, and/or events. The local reference marker 126 may be a reference line or a cursor. The operator may move the local reference marker 126 using a pointing device, such as, but not limited to, a mouse, a knob, or the operator's finger on a touch-screen. As the local reference marker 126 passes over the graphics, data, for example, the numerical data 128 appears on the display 111 showing, for example, critical values, alarms, and system changes for that given moment in the local timeline 116.

In an exemplary embodiment, the operator controls the duration of time being reviewed. For example, the operator may select a shorter period of time to review the information in greater detail. Alternatively, the operator may choose to review a longer period of time to see long term trends. Historic trending enables an operator to quickly scan a patient's history and keep track of critical or important numeric indicators of patient status.

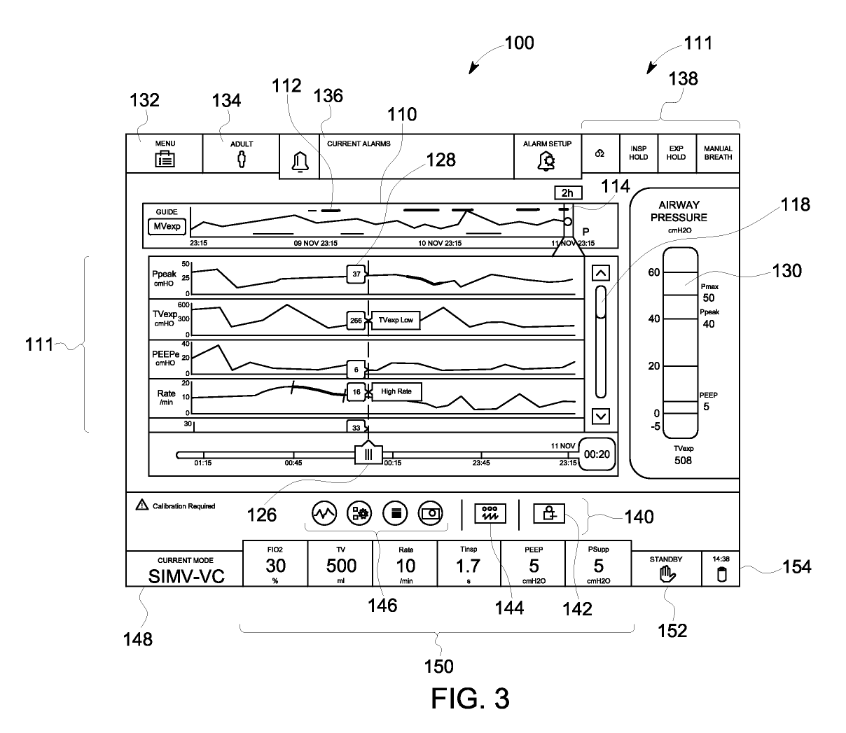

FIG. 3 is an exemplary view of a screen 102 including the patient ventilator parameter display 111. The screen 102 displays a visualization of patient ventilator parameters. In the illustrated embodiment, a scale 130 may be displayed with the display 111. The scale 130 represents an airway pressure of the patient. In one embodiment, the scale 130 may be toggled on and off by an operator. The scale 130 may be provided, for example, as described in copending application Ser. No. 13/112,870, entitled “METHOD AND SYSTEM FOR VISUALIZING VENTILATION INFORMATION”, and commonly owned.

A menu button 132 is provided for selecting a menu, for example, a menu screen or drop down menu that enables the operator to update and/or change various parameters of the screen 102 or to access, for example, system settings, as well as configuring and running different procedures. A patient button 134 is provided for selecting a type of patient, for example, adult, child, or infant. Various operating parameters may be updated based on the type of patient. Additionally, various compliance ranges may updated based on the type of patient. An alert screen button 136 is activated to display current alert, notifications. The alert notifications may also be accompanied by visual and/or audible alarms; In one embodiment, the alert screen button 136 may activate a drop down screen that displays the most recent alerts. Function buttons 138 are provided to instruct the ventilator to perform various functions. It should be noted that although the various buttons are shown as user selectable soft keys (e.g. virtual buttons displayed on the screen), the buttons may be any type of hard or soft button, key, etc. Additionally, instead or in addition to buttons, any type of user interaction control or input, whether virtual or physical may be provided.

View buttons 140 are provided to change an appearance of the screen 102, view a present screen, or view a future screen. For example, by selecting a future screen button 142, a future screen is displayed illustrating future patient ventilator parameters. By selecting a present screen button 144, a present screen is displayed illustrating present patient ventilator parameters. Additionally, multiple past screen buttons 146 are provided to change a view or format of the screen 102. The operator may toggle between the screen 102, a present screen, and/or a future screen, by selecting the corresponding view buttons 140. Additionally, the operator may toggle between the various views and formats of the screen 102 by selecting the corresponding view buttons 140. For example, the screen 102 may be displayed in various graphical or numerical formats.

A mode button 148 may be selected to change and/or update an operating mode of the ventilator. Parameter buttons 150 display desired compliance levels for various parameters. The parameter buttons 150 may he selected to alter the characteristics of the ventilation that is provided to the patient. A standby button 152 may be selected to pause the operation of the ventilator. Operation of the ventilator may be paused during various patient treatments, for system calibration, or the like. A battery display 154 indicates a battery level of at least one of the monitor 38 and/or the ventilator 16 (both shown in FIG. 1).

It should be noted that although the embodiments are described with respect to various functional buttons, not all of the functional buttons are required to practice the embodiments described herein. Additionally, various other functional buttons may be included on the screen 102.

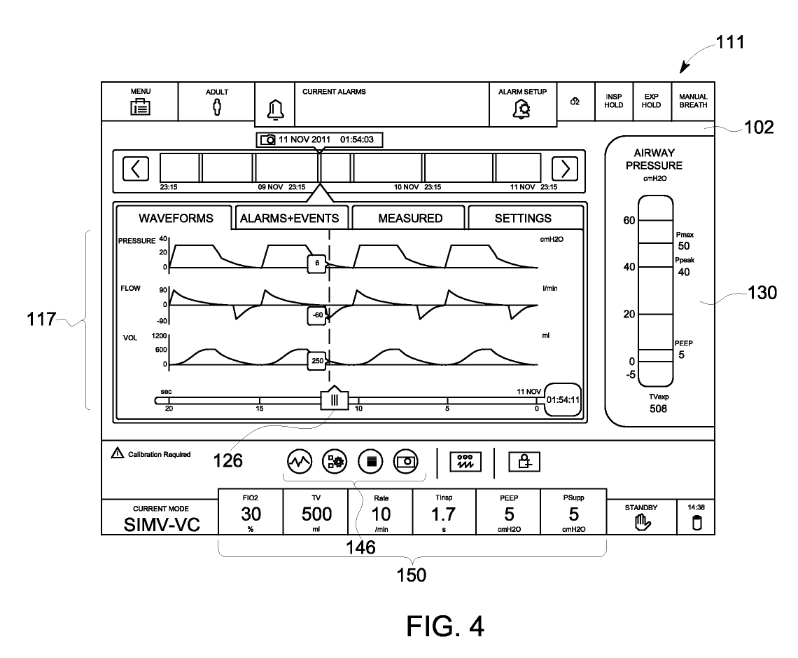

FIG. 4 is another exemplary view of the screen 102. FIG. 4 illustrates waveforms 117 representative of a snapshot of a patient ventilator parameter. An operator may choose the format of the screen 102 illustrated in FIG. 4 by selecting one of past screen buttons 146. The operator may toggle between the view illustrated in FIG. 4 and other views using the past screen buttons 146 while maintaining the same time reference as described in more detail herein. The waveforms shown in FIG. 4 enable the operator to view a snapshot of data from the timelines 116 (shown in FIG. 2). As illustrated in FIG. 4, the buttons and controls described in FIG. 3 arc still available to the operator. Additionally, the local reference marker 126 is provided to select data within the waveforms.

The master timeline 110 in FIG. 4 illustrates an alternate means of displaying patient status. In particular, event reference markers 119 (shown s vertical bars) that mark times, for example, for important or significant events in the status or treatment of the patient. An operator may select an event of interest in the master timeline 110 to view a snapshot of the patient status and ventilator setting parameters at that time. Thus, a clinician is able to take a “snapshot” of all the current settings and patient status, which then may be later selected and displayed to the operator. In some embodiments, the system automatically marks times of particular events, such as significant events (e.g., large changes in settings or patient status). For example, the system may indentify potential times of interest (for the clinician to investigate) based on an automated assessment of one or more measurements and/or system states.

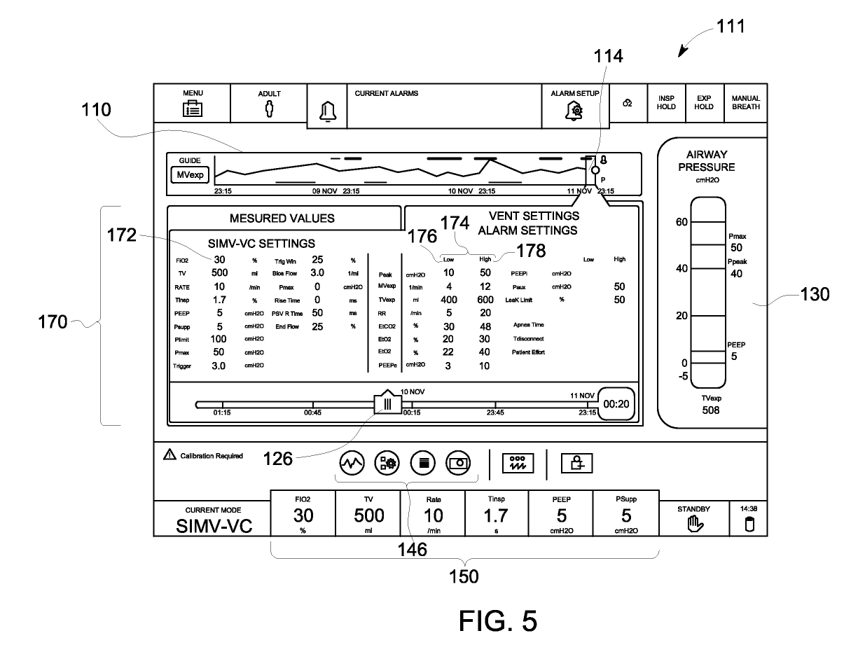

FIG. 5 is another exemplary view of a screen 102. FIG. 5 illustrates a ventilator setting and measured values chart 170. The operator may toggle between the chart 170 and other views using the past screen buttons 146. The chart 170 numerically displays the values 172 of the various patient ventilator settings, which determine the characteristics of the ventilation provided, as well as the alarm settings (also referred to herein as compliance ranges). Thus, the chart 170 may also display the compliance ranges 174 for alerts and alarms. The compliance ranges 174 include a low value 176 and a high value 178. An alert is triggered if a value of a parameter falls outside of the compliance range 174 by either falling below the low value 176 or exceeding the high value 178. The master timeline 110 is displayed with the chart 170. A local reference marker 126 is also displayed with the chart 170. By moving the local reference marker 126, an operator can view the measured values of the parameter at a time corresponding to the position of the local reference marker 126. As the local reference marker 126 is moved, the values 172 and the compliance ranges 174 are updated accordingly. The values 172 may correspond to different measured parameters or settings as desired or needed.

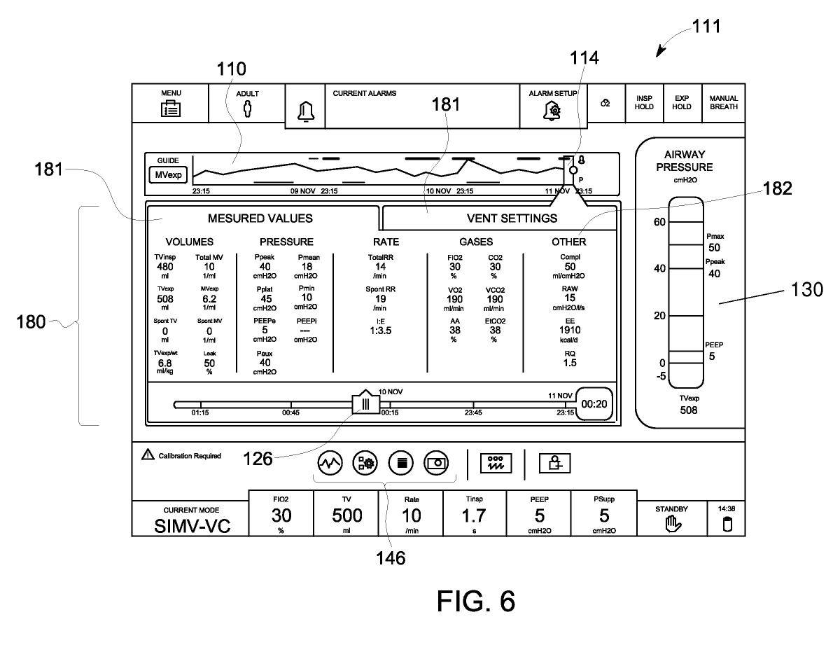

FIG. 6 is another exemplary view of a screen 102. FIG. 6 illustrates a measured patient values chart 180. This interface generally provides a tabbed view to allow switching between measured values and ventilator settings by selecting the corresponding tab 181. The operator may also toggle between the chart 180 and other views using the past screen buttons 146. The chart 180 numerically selectably displays the values 182 of the various measured values (or ventilator parameters). The master timeline 110 is displayed with the chart 180. A local reference marker 126 is also displayed with the chart 180. By moving the local reference marker 126, an operator can view the measured values 182 of ventilator parameters at a time corresponding to the position of the local reference marker 126. As the local reference marker 126 is moved, the values 182 are updated accordingly.

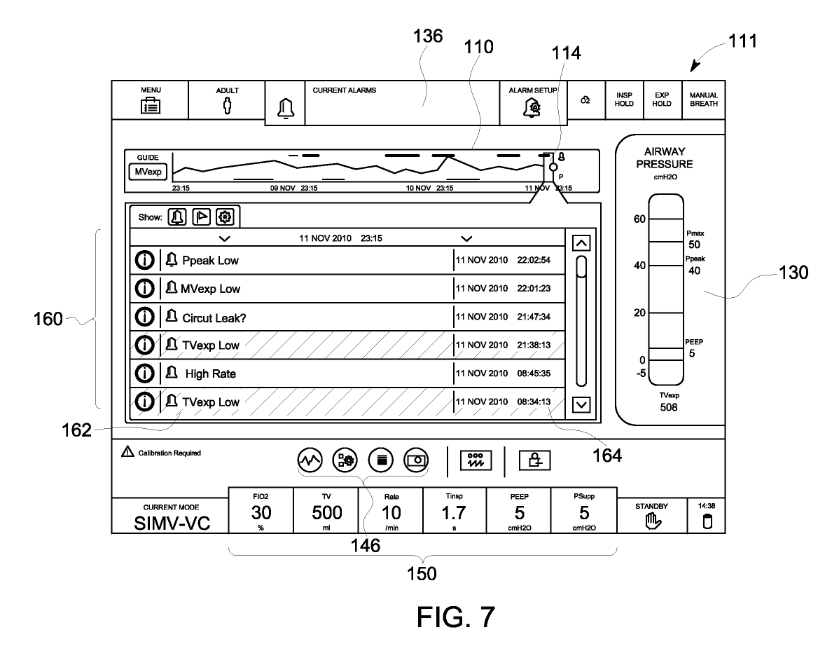

FIG. 7 is another exemplary view of a screen 102. FIG. 7 displays an alert and event log screen 160 that may be accessed by selecting the button 136. The alert and event log screen 160 displays all of the alerts throughout the time that the patient has been ventilated. The alert and log screen 160 can also show any significant patient or ventilator related events, such as specific procedures that are run, ventilator setting changes, clinician identified times of interest (snapshots) and system identified times of interest, among others. Thus, a chronological log of significant events may he displayed. The events generally include alarms, but may also include or support logging when specific procedures were run (and the results) and when the ventilator setting changes were made (e.g., changing the ventilation modes). Accordingly, once a clinician has identified a time period of interest, for example, when observing a dramatic change in readings in the graphical trend view), the alert and log screen 160 may be accessed to view any significant events that occurred immediately before or after that time. For example, the events may include a change in ventilation mode or a procedure that was run that caused the change in patient status.

Alternatively, the alert screen 160 may be configured to display only the alerts within a determined time range. The alert and event log screen 160 includes a scroll bar to scroll through the displayed alerts. The alert and event log screen 160 includes a parameter indicator 162 identifying parameters that fell outside of the compliance range. The alert and event log screen 160 also includes time indicators 164 displaying the time and date that a corresponding alert took place. An operator may select one of the parameter indicators 162 to obtain further information regarding the alert, for example, a parameter level at the time of the alert.

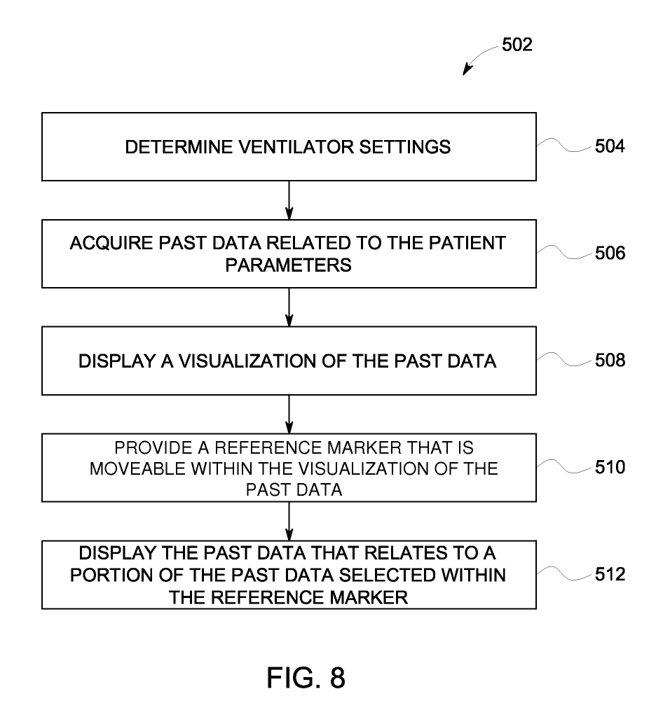

FIG. 8 is a flowchart illustrating a method 502 for displaying data from a ventilator, for example, the ventilator 16 (shown in FIG. 1). In one embodiment, instructions may be stored on a non-transitory computer readable storage medium to command a processor, for example, the processor 58 (shown in FIG. 1) to perform the method 502. At 504, ventilator settings are determined. For example, the operator may select various ventilator settings to configure how the patient is ventilated. In one embodiment, compliance ranges may be selected for measured patient data. The compliance ranges define a high and low value for different measured parameters. If a value of the parameter falls below the low value or exceeds the high value, an alert notification is triggered. The compliance range may be manually selected by the operator. Alternatively, predetermined compliance ranges may be selected.

At 506, past data related to the patient parameters is acquired (and recorded or stored). At 508, a visualization of the past patient and ventilator parameters is displayed over an extended period of time. For example, the past patient parameters may be displayed on a timeline or other similar graphical representation, as described herein. At 510, a reference marker is provided on the visualization of the past patient parameters. For example, the reference marker may be a reference line that is moveable along a timeline representing the past patient parameters. The reference marker may be utilized to select a portion of the past patient parameters representative of a time segment over the extended period of time. At 512, data from the past patient and ventilator parameters is displayed based on the portion of the past patient and ventilator parameters selected with the reference marker.

In various embodiments, “ventilation settings” or “ventilator settings” generally refers to parameters that determine or define how ventilation is provided to the patient. These settings are parameters selected by a clinician. In various embodiments, measured values and data, such as patient parameters, generally refer to the reading acquired from the sensors 34 (shown in FIG. 1 and which may be connected to the patient directly or indirectly) that represent the status of the patient. It should be noted that some quantities can be both a setting and a measurement, and some quantities may be either depending on the current ventilation mode. It should be noted that compliance ranges are also generally settings (or alarm settings).

In one embodiment, the method 502 includes storing one or more sensor measurements related to the patient status. A visualization of past patient measurements is displayed. A reference marker is displayed to select a portion of the past patient measurements. Data from the past patient measurements is displayed based cm the portion of the past patient measurements selected with the reference marker. The past patient measurements may be displayed on a timeline. The reference marker is displayed as a reference line that is moveable along the timeline. The past patient measurements may also be displayed on a master timeline. A portion of the past patient measurements is displayed on a timeline that represents a portion of the master timeline. In one embodiment, the past patient measurements are displayed on a touch screen display. A portion of the past patient measurements is selected by manipulating the reference marker with the touch screen display. In one embodiment, alerts are displayed on the visualization of past patient measurements. The alerts represent a patient measurement falling outside of a compliance range for that parameter. In one embodiment, a scale representing an airway pressure of the patient is displayed with the past patient measurements. In one embodiment, the past patient measurements are monitored so that the characteristics of the patient fall within a compliance range. In one embodiment, the past parameters are displayed in at least one of a graphical or numerical format.

Thus, various embodiments provide for visualization of ventilator related information or data, such as the display of past data on a timeline having a reference line to select data points on the timeline. The visualizations may be numerical, graphical or a combination thereof. Additionally, the various visualizations and displayed indicators may be modified as desired or needed, for example, based on user preferences or system settings.

Exemplary embodiments of a medical system with a ventilator are described above in detail. The components illustrated are not limited to the specific embodiments described herein, but rather, components of the system may be utilized independently and separately from other components described herein. For example, the medical system components described above may also be used in combination with other medical systems, such as medical imaging or diagnostic systems.

It should be noted that the various embodiments, for example, the modules described herein, may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive, optical disk drive, solid state disk drive (e.g., flash drive of flash RAM) and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software. Further, the software may be in the form of a collection of separate programs, a program module within a larger program or a portion of a program module or a non-transitory computer readable medium. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM Memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A medical system having a ventilator, the medical system comprising:
a memory storing data related to patient and ventilator parameters;
a processor programmed to organize, in time, the data related to the patient and ventilator parameters; and
a viewer to:
display the patient and ventilator parameters for an overall monitoring time period on a timeline,
display a reference marker on the timeline selecting a segment of the patient and ventilator parameters corresponding to a past monitoring time, wherein the segment selected represents a time segment within the overall monitoring time period, and
display the patient and ventilator parameters corresponding to the time segment.

2. The medical system of claim 1, wherein the viewer further:
displays the patient and ventilator parameters over the overall monitoring time period on a master timeline; and
displays the reference marker as a master reference marker that is moveable along the master timeline.

3. The medical system of claim 1, wherein the viewer further:
displays the patient and ventilator parameters over the overall monitoring time period on a master timeline; and
displays the patient and ventilator parameters corresponding to the time segment on a local timeline.

4. The medical system of claim 1, wherein the viewer further:
displays the patient and ventilator parameters corresponding to the time segment on a local timeline; and
displays a local reference marker that is moveable along the local timeline to select data points on the local timeline.

5. The medical system of claim 1, wherein the viewer further displays the patient and ventilator parameters corresponding to the time segment in at least one of a graphical or numerical format.

6. The medical system of claim 1, wherein the viewer is a touch screen display, the patient and ventilator parameters corresponding to the time segment selectable by manipulating the reference marker with the touch screen display.

7. The medical system of claim 1, wherein the viewer further displays (i) alerts representing a patient parameter falling outside of a compliance range for the parameter or (ii) significant patient or ventilator related events.

8. The medical system of claim 1, wherein the viewer further displays the patient and ventilator parameters corresponding to the time segment in multiple formats, the timeline associated with the overall monitoring time period displayed with each format such that the time segment defining a selected time reference is maintained when the multiple formats are displayed.

9. A method for presenting ventilator data, the method comprising:
storing data related to patient and ventilator parameters;
organizing, in time, the data related to the patient and ventilator parameters:
displaying the patient and ventilator parameters for an overall monitoring time period on a timeline;
displaying a reference marker on the timeline selecting a segment of the patient and ventilator parameters corresponding to a past monitoring time, wherein the segment selected represents a time segment within the overall monitoring time period; and
displaying the patient and ventilator parameters corresponding to the time segment.

10. The method of claim 9 further comprising:
displaying the patient and ventilator parameters over the overall monitoring time period on a master timeline; and
displaying the reference marker as a master reference marker that is moveable along the master timeline.

11. The method of claim 9 further comprising:
displaying the patient and ventilator parameters over the overall monitoring time period on a master timeline; and
displaying the patient and ventilator parameters corresponding to the time segment on a local timeline.

12. The method of claim 9 further comprising:
displaying the patient and ventilator parameters corresponding to the time segment on a local timeline; and
displaying a local reference marker that is moveable along the local timeline to select data points on the local timeline.

13. The method of claim 9 further comprising displaying the patient and ventilator parameters corresponding to the time segment in at least one of a graphical or numerical format.

14. The method of claim 9 further comprising selecting the patient and ventilator parameters corresponding to the time segment by manipulating the reference marker with a touch screen display.

15. The method of claim 9 further comprising displaying alerts representing (i) a patient ventilator or parameter falling outside of a compliance range for the parameter or (ii) a significant patient or ventilator related event.

16. The method of claim 9 further comprising:
displaying the patient and ventilator parameters corresponding to the time segment in multiple formats; and
displaying the timeline associated with the overall monitoring time period with each format.

17. A non-transitory computer readable storage medium for displaying ventilator information using a processor, the non-transitory computer readable storage medium including instructions to command the processor to:
store data related to patient and ventilator parameters;
organize, in time, the data related to the patient and ventilator parameters;
display the patient and ventilator parameters for an overall monitoring time period on a timeline;
display a reference marker on the timeline selecting a segment of the patient and ventilator parameters corresponding to a past monitoring time, wherein the segment selected represents a time segment within the overall monitoring time period; and
display the patient and ventilator parameters corresponding to the time segment.

18. The non-transitory computer readable storage medium of claim 17, wherein the instructions command the processor to:
display the patient and ventilator parameters over the overall monitoring time period on a master timeline; and
display the reference marker as a master reference marker that is moveable along the master timeline.

19. The non-transitory computer readable storage medium of claim 17, wherein the instructions command the processor to:
display the patient and ventilator parameters over the overall monitoring time period on a master timeline; and
display the patient and ventilator parameters corresponding to the time segment on a local timeline.

20. The non-transitory computer readable storage medium of claim 17, wherein the instructions command the processor to:
display the patient and ventilator parameters corresponding to the time segment on a local timeline; and
display a local reference marker that is moveable along the local timeline to select data points on the local timeline.
